(19) United States
(12) Patent Application Publication
(10) Pub. No.: US 2006/0223998 A1
(43) Pub. Date: Oct. 5, 2006

(54) Synthetic LSD metabolite for preparing haptens used in an LSD metabolite immunoassay

(76) Inventors: Fang-Jie Zhang, Sunnyvale, CA (US); Hshiou-ting Liu, Milpitas, CA (US)

Correspondence Address: DADE BEHRING INC.;LEGAL DEPARTMENT, 1717 DEERFIELD ROAD, DEERFIELD, IL, 60015, US

(21) Appl. No.: 11/093,676

(22) Filed: Mar. 30, 2005

(51) Int. Cl. C07D 457/02

(52) U.S. Cl. 546/69

(57) ABSTRACT

A general method for the synthesis of OH-LSD and its analogs has been successfully developed. This method is convenient and efficient and allows preparation of a series of OH-LSD analogs with linkers attached at various (C-8, N-6 or N-1) positions.

### FIELD OF THE INVENTION

The present invention relates generally to detection of drugs and drug metabolites in biological samples. More specifically, it provides a method for synthesizing a metabolite for LSD useful for confirming the presence of LSD in a sample.

### BACKGROUND OF THE INVENTION

Lysergic acid diethylamide (LSD) is a highly potent hallucinogen first synthesized in 1938 and its psychotic effect was observed in 1943 as the result of an accidental ingestion. In the 1950s-1960s, psychiatrists conducted clinical experimentation with LSD to treat mental illness. However, at the same time, non-medical use of LSD was popular due to its hallucinogenic effect. Since 1966, LSD has been an illegal drug. According to a survey on drug abuse, LSD consumption has increased since the beginning of the 1990s after a drop in the 1980s.

LSD undergoes rapid and extensive metabolism, with only about 1% of the parent drug being found in human urine. Possible metabolic transformations may be hydrolysis to lysergic acid, N-demethylation to N-desmethyl-LSD and/or oxidation to 2-oxo-LSD and 2-oxo-3-hydroxy-LSD. Isolysergic diethylamide (iso-LSD) is a byproduct of LSD synthesis and is often detected in the urine from an LSD user because of its presence as a contaminant in LSD.

LSD is one difficult to detect in urine because of the very low concentrations of the parent drug found in urine. 2-Oxo-3-hydroxy-LSD is a recently identified metabolite of LSD that has been found to be present in urine from LSD users at concentrations from 4 to 40 times higher than LSD and that can be detected for a longer time than LSD after ingestion of the drug.

Factors that have contributed to continued LSD use are wide availability, low cost, and the difficulty of detecting LSD use by analysis of body fluids. The usual oral dose of LSD is only 20-80 μg (ref. 3), and the drug is extensively metabolized in the liver. Less than 1% of the initial LSD is eliminated unchanged in urine. Conclusive identification of LSD in body fluids remains a challenging analytical task.

In testing for other drugs of abuse, immunoassays, particularly competitive binding immunoassays, have proven to be especially advantageous. In competitive binding immunoassays, an analyte in a biological sample competes with a labeled reagent, or analyte analog, or tracer, for a limited number of receptor binding sites on antibodies specific for the analyte and analyte analog. Enzymes such as β-galactosidase and peroxidase, and fluorescent molecules such as flurescein compounds, are common labeling substances used as tracers. The concentration of analyte in the sample determines the amount of analyte analog which will bind to the antibody. The amount of analyte analog that will bind is inversely proportional to the concentration of analyte in the sample, because the analyte and the analyte analog each bind to the antibody in proportion to their respective concentrations. The amount of free or bound analyte analog can then be determined by methods appropriate to the particular label being used.

Recently it is reported that one of LSD metabolites, 2-oxo-3-hydroxy-LSD (OH-LSD), could be used as a better target molecule for the detection of LSD use. Two research groups showed that on average, the concentration of OH-LSD to LSD was at least 20-fold higher than the concentration of LSD. Furthermore, within 24-36 hours after drug ingestion, the concentration of LSD fell below the cutoff concentration (200 Pg/mL), while OH-LSD concentration was above the cutoff concentration for up to 48 h post-administration. The consistent and relatively large concentration of OH-LSD in body fluids shows great promise for more effective detection of LSD exposure by testing for OH-LSD instead of LSD. To develop a new assay method based on screening OH-LSD, there was a need for the synthesis of a series of OH-LSD derivatives.

OH-LSD was first synthesized in 1959 by oxidation of LSD with calcium hypochlorite in about 30% yield. In 1989, an OH-LSD analog was prepared by treatment of α-ergocryptine, a LSD analog, with a solution of hydrogen bromide in DMSO in about 50% yield (ref 8). However, no conclusive data were available to support the structure of the product. In 1994, another LSD analog was converted to OH-LSD analog with bromine in the presence of water in about 50% yield. However, to date no general method for the synthesis of OH-LSD analogs has been reported.

U.S. Pat. No. 6,794,496 provides hapten derivatives useful for the preparation of antigens, antibodies and reagents for use in immunoassays for the detection of LSD and 2-oxo-3-hydroxy LSD. In the present invention, the 2-oxo-hydroxy-3-LSD nucleus is derivatized out of the indole nitrogen to form an aminoalkyl derivative. The resulting haptens can then be further modified at this functionalized position for linking to appropriate immunogenic or labeling groups to provide reagents for immunoassays having substantially equal specificity for both LSD and 2-oxo-3-hydroxy-LSD.

U.S. Pat. No. 6,794,496 discloses compositions and methods applicable for generating antibodies specific for a LSD metabolite, 2-oxo-3-hydroxy-LSD, or its derivatives. This invention also provides the uses of these antibodies for the detection or measurement of LSD or 2-oxo-3-hydroxy-LSD in samples obtained from subjects who may have been exposed to LSD. In various embodiments, the system allows for detection of both the parent substance and natural metabolites as they may be formed within the subject or secreted into a biological fluid, particularly urine. The sensitivity and specificity of the reagents may be used in diagnostic-grade immunoassays for screening of drugs of abuse in a clinical setting.

U.S. Pat. No. 6,207,396 discloses reagents for use in LSD immunoassays involving the coupling to or derivatization of the maleimide modified activated hapten precursor compound via sulfhydryl groups on an immunogenic carrier substance. The immunogens of the present invention, which include the haptenic drug covalently linked via its maleimide moiety and a sulthydryl bridge to an immunogenic carrier material, are used to stimulate the production of antibodies to LSD.

### SUMMARY OF THE INVENTION

To improve the monitoring of LSD usage in drug-of-abuse immunoassay testing, a new procedure has been developed for the synthesis of a major LSD metabolite, 2-oxo-3-hydroxy-LSD (OH-LSD), and its derivatives. The concentration of OH-LSD in the human body fluids is relatively high compared to that of LSD and other metabolites. Unlike LSD itself the concentration of this particular metabolite, instead of LSD itself, remains relatively constant for several days and offers a prolonged diagnostic window for potential LSD exposure. Therefore, OH-LSD appears to be a promising candidate molecule for monitoring LSD use. However, the availability of OH-LSD has been a problem due to the tedious process involved in the extraction and purification from the biological fluids, and the lack of a reliable method for its synthesis. The present invention is a novel and ready synthesis of OH-LSD and its analogs for the development of a LSD metabolite assay.

### BRIEF DESCRIPTION OF THE DRAWINGS

The invention will be more fully understood from the following detailed description thereof taken in connection with the accompanying drawings which form a part of this application and in which:

FIG. 1 shows LSD, its major metabolite OH-LSD and the transformation therebetween;

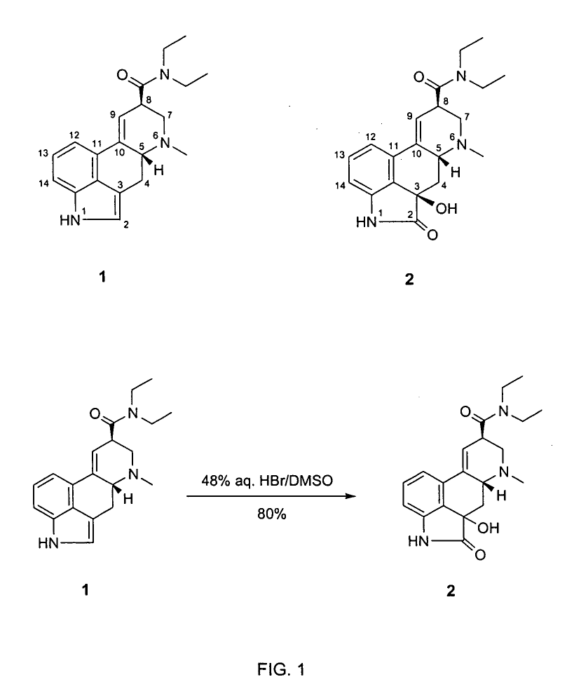

FIG. 2 shows the synthesis of OH-LSD analogs with a link at the C-8 position;

FIG. 3 shows the synthesis of OH-LSD analogs with a link at the N-6position;

FIG. 4 shows the synthesis of OH-LSD analogs with a link at the N-1 position; and,

FIG. 5 shows the OH-LSD analogs with links at the C-8, N-6 and N-1positions reacted with KLH protein to give three respective immunogens.

### DETAILED DESCRIPTION OF THE PREFERRED EMBODIMENTS

The major objective of the present invention is the development of a new and general method to the synthesis OH-LSD and its analogs. The newly synthesized OH-LSD analogs may be advantageously conjugated to proteins to prepare immunogens and enzyme-hapten conjugates for monitoring LSD consumption.

Synthetic Method to OH-LSD

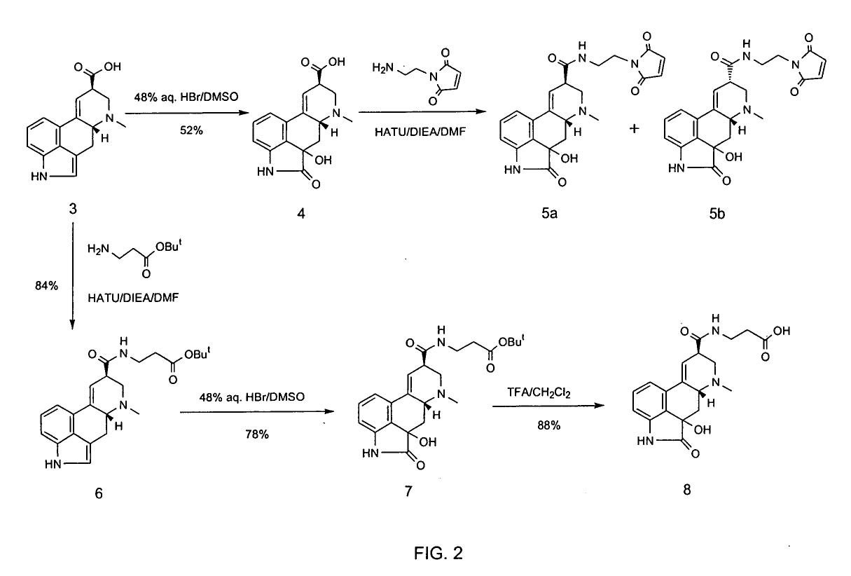

Considering the possible mechanism in which the LSD analogs were transformed to their OH-analogs in biological system, it has been discovered that 2-bromo- or 2-oxo-LSD may be the reaction intermediate which will be further oxidized to the OH-analogs and water is required for the transformation. Accordingly, a new reaction procedure has been developed for the conversion of LSD to OH-LSD using an aqueous HBr/DMSO reagent system (as seen in FIG. 2). Briefly, to a solution of LSD in DMSO was added hydrobromic acid (48% in water) drop-wise at room temperature. The mixture was stirred at room temperature for 70 min. Then water was added and the mixture was stirred for another 30 min. After a conventional workup, OH-LSD was obtained in about 80% yield. It has been found that excess amount of DMSO and HBr (12 eq.) is a critical factor for maintaining a high yield and that water is also necessary for the reaction. When hydrobromic acid was replaced by hydrogen bromide solution in acetic acid, OH-LSD was obtained in a very low yield.

Synthesis of OH-LSD Analogs with a Link at C-8 Position

The present invention was then employed for the successful synthesis of OH-lysergic acid (4) from lysergic acid (3) (also seen in FIG. 2). Reaction of compound 4 with 1-(2-aminoethyl)-1H-Pyrrole-2,5-dione in the presence of HATU and diisopropylethylamine in DMF afforded its maleimide derivatives (compounds 5a and 5b). Compounds 5a and 5b are diastereomers at C-8 position. Reaction of lysergic acid with β-alanine t-butyl ester furnished compound 6, which was then treated with aqueous HBr/DMSO system to give compound 7 in a good yield (78%). Acidolysis of compound 7 with trifluoroacetic acid in CH₂Cl₂ gave compound 8, a free carboxylic acid analog of OH-LSD.

Synthesis of OH-LSD Analogs with a Link at N-6 Position

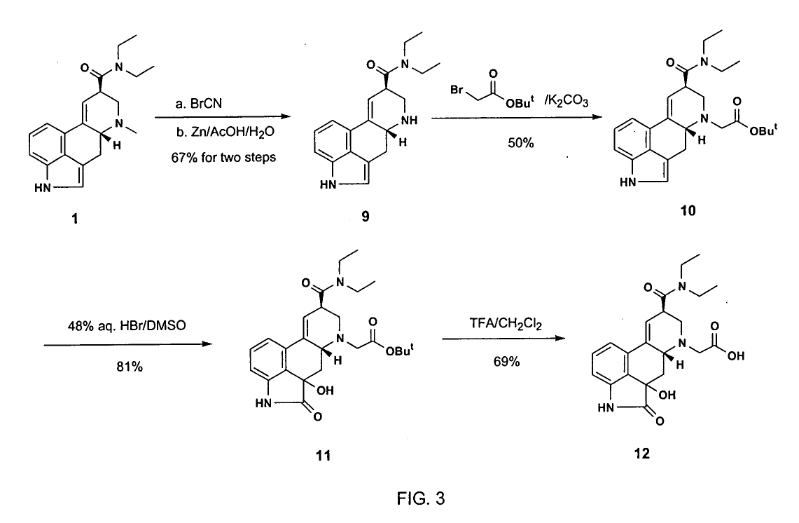

This synthesis started with nor-LSD, compound 9, which was prepared by demethylation of LSD in two steps (FIG. 3). Compound 9 reacted with t-butyl-2-bromoacetic ester in the presence of potassium carbonate in DMF to give a mixture of two diastereomers, in which compound 10 is the major product. Compound 10 was subjected to the aqueous HBr/DMSO system to give the desired compound 11 at an 81% yield. After acidolysis with TFA, an OH-LSD analog 12 with a link at the N-6 position was obtained.

Synthesis of OH-LSD Analogs with a Link at the N-1 Position

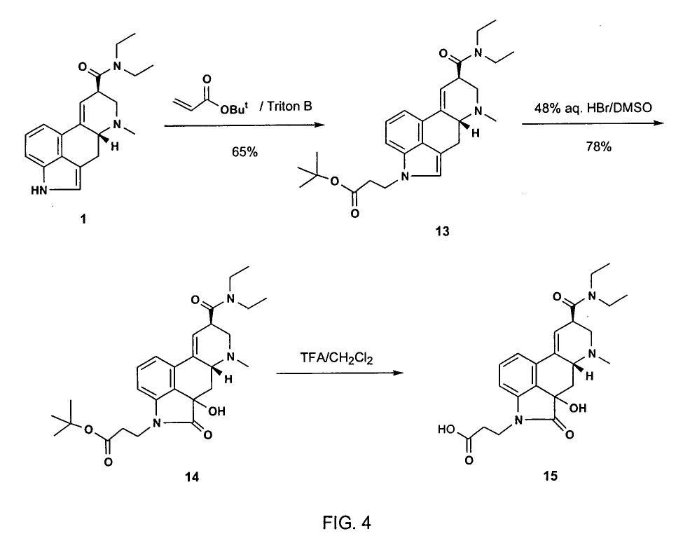

As outlined in FIG. 4, LSD was alkylated to give 13 by Michael addition reaction with t-butyl acrylate in the presence of Triton B. Compound 13 was treated with aqueous HBr/DMSO to afford compound 14 in good yield. After acidolysis, the desired compound 15 was obtained.

Preparation of Immunogens for an LSD Immunoassay

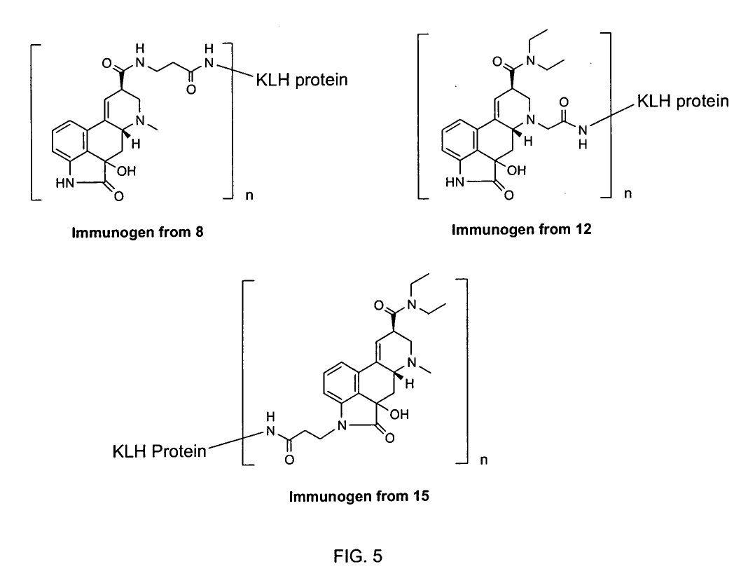

OH-LSD analogs with a link at C-8 (compound 8), N-6 (compound 12) and N-1 (compound 15) were respectively transformed to their N-hydroxysuccinimide esters, which then reacted with KLH protein to give the three immunogens as shown in FIG. 5.

A general method for the synthesis of OH-LSD and its analogs has been successfully developed. This method is convenient and efficient and allows preparation of a series of OH-LSD analogs with linkers attached at various (C-8, N-6 or N-1) positions. These new compounds are useful in the preparation of immunogens for the development of a LSD metabolite assay.

It should be readily understood by those persons skilled in the art that the present invention is susceptible of a broad utility and application. Many embodiments and adaptations of the present invention other than those herein described, as well as many variations, modifications and equivalent arrangements will be apparent from or reasonably suggested by the present invention and the foregoing description thereof, without departing from the substance or scope of the present invention.

Accordingly, while the present invention has been described herein in detail in relation to specific embodiments, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements, the present invention being limited only by the claims appended hereto and the equivalents thereof.

1-3. (canceled)

4. A method to prepare LSD-OH or an analog of LSD-OH comprising:
(a) adding an aqueous solution of hydrobromic acid to LSD or an analogue of LSD in DMSO;
(b) purifying the LSD-OH or analog of LSD-OH; and wherein the LSD analog is derivatized at position C-8, N-6 or N-1.

5. The method of claim 4 wherein the purified LSD-OH or analog LSD-OH is further reacted with an immunogenic carrier.

6. The method of claim 5 wherein the immunogenic carrier is KLH.

7. The method of claim 4 wherein the hydrobromic acid is at least 12 equivalents.

8. The method of claim 4 wherein the LSD is derivatized at position C-8, N-6 or N-1.

9. The method of claim 4 wherein the LSD analog is selected from the group consisting of lysergic acid and nor-LSD.

10. The method of claim 4 wherein the yield of the LSD-OH or analog thereof is greater than about 80%.

11. The method of claim 8 wherein the yield is 78%.

12. The method of claim 8 wherein the purified LSD-OH derivative or analog LSD-OH derivative is further reacted with an immunogenic protein.

13. An intermediate for the preparation of immunogens to LSD or LSD-OH prepared by the process comprising:
(a) providing an aqueous solution of hydrobromic acid, DMSO and LSD or an analogue of LSD derivatized at positions C-8, N-6 or N-1; and
(b) purifying the LSD-OH or derivatized analog of LSD-OH.
